(19) United States
(12) Patent Application Publication
(10) Pub. No.: US 2014/0283847 A1
(43) Pub. Date: Sep. 25, 2014

(54) NEGATIVE PRESSURE WOUND TREATMENT DRESSINGS AND SYSTEMS

(71) Applicant: KCI Licensing, Inc., San Antonio, TX (US)

(72) Inventors: T. Blane Sanders, San Antonio, TX (US); Robert P. Wilkes, San Antonio, TX (US)

(21) Appl. No.: 14/297,234

(22) Filed: Jun. 5, 2014

Related U.S. Application Data
(62) Division of application No. 12/730,582, filed on Mar. 24, 2010.
Application No. 12/730,582 is a continuation-in-part of application No. 10/895,700, filed on Jul. 21, 2004, now Pat. No. 8,444,611.
(60) Provisional application No. 60/489,344, filed on Jul. 22, 2003.

(51) Int. Cl. A61M 1/00; A61B 19/12

(52) U.S. Cl. CPC A61M 1/0088; A61B 19/12; A61B 2019/085
USPC 128/853; 29/428

(57) ABSTRACT

Dressings, systems, and methods to facilitate the application of reduced pressure to an extremity, such as heel, on a patient are provided. In one instance, a dressing for treating an extremity on a patient includes a drape envelope, a transport manifold, and a pouch. The drape envelope has an outer drape member, inner drape member, and an interior portion between the outer drape member and the inner drape member. The inner drape member has fenestrations on at least an application portion. The transport manifold is disposed within the interior portion of the drape envelope. The pouch is for receiving the extremity and includes the drape envelope and a pouch-forming attachment. The dressing allows reduced pressure to be initially supplied at a remote location from the wound and avoid leaks. Other systems, methods, and apparatuses are disclosed.

### RELATED APPLICATIONS

This application is a divisional of U.S. patent application Ser. No. 12/730,582, filed Mar. 24, 2010, which is a continuation-in-part of U.S. patent application Ser. No. 10/895,700, filed Jul. 21, 2004, which claims the benefit of U.S. Provisional Application No. 60/489,344, filed Jul. 22, 2003, the entirety of each of which are hereby incorporated by reference for all purposes.

### FIELD

The embodiments herein relate to the treatment of open, chronic wounds, as well as burns and skin grafts. More specifically the embodiments relate to dressings, methods, and systems for treating wounds on the extremities, such as the heel of a human foot, or other limited-access wound sites.

### BACKGROUND

Negative pressure wound therapy has been utilized for the treatment of open wounds and has been commercialized by Kinetic Concepts, Inc. of San Antonio, Tex., by its proprietary V.A.C.® medical device product line. In practice, the application to a wound of negative gauge pressure, commercialized by Kinetic Concepts, Inc. under the designation “Vacuum Assisted Closure®” (or “V.A.C.®”) medical device therapy, typically involves the mechanical-like contraction of the wound with simultaneous removal of excess fluid, which is often accomplished by means of a polymer foam and occlusive drape dressing in fluid communication with a negative pressure source. In this manner, negative pressure wound therapy augments the body's natural inflammatory process while alleviating many of the known intrinsic side effects, such as the production of edema caused by increased blood flow absent the necessary vascular structure for proper venous return. As a result, negative pressure wound therapy has been highly successful in the promotion of wound closure, healing many wounds previously thought largely untreatable.

Many of these wounds include decubitus and venous stasis ulcers to the lower extremities, especially the foot. Closure of these wounds has been difficult and often times impossible using traditional techniques, such as skin grafting, sharp debridement, or combinations thereof. Failure to close these wounds, which have often been present for several years, can lead to necrotizing of the tissue, and in many cases amputation of the extremity. Use of negative pressure wound therapy has proven highly successful in closing these wounds. However, treatment of the lower extremities with negative pressure wound therapy, especially to wounds of the foot and heel can be difficult, especially considering the nature of the location of the wound. Particular concern arises with maintaining the dressing on the extremity, especially in light of the frequent movement of the foot, and friction often associated with foot coverings, including socks, stockings, and shoes. Of particular concern, is the ability to maintain a negative pressure at the wound site when the dressing is in place, as air leaks may occur during movement of the foot, which can adversely affect the therapy being administered.

For the foregoing reasons, there is a need for a negative pressure wound treatment dressing system that is capable of maintaining a negative pressure over an extremity that has heretofore been difficult to treat using traditional negative pressure wound therapy dressings. Additionally, there is a need for a negative pressure wound treatment dressing system that is capable of maintaining a negative pressure over a wound which exists in a highly contoured part of the body, or a portion of the body that undergoes frequent movement or friction against clothing or other outside forces.

It is therefore an object of the present invention to provide a negative pressure wound treatment dressing system that provides a means for maintaining a negative pressure over a contoured extremity, such as the foot, and more specifically the heel of the foot.

### SUMMARY

Problems with medical treatment systems, devices, and methods are addressed by the negative pressure systems, dressings, and methods of the illustrative, non-limiting embodiments described herein. According to one illustrative, non-limiting embodiment a dressing for treating an extremity of a patient with reduced pressure includes a transport manifold and a drape envelope. The transport manifold includes a receiving site, an extension portion, and an application portion. The drape envelope includes an outer drape member and a second, patient-facing drape member. The drape envelope also has an interior portion between the outer drape member and the second, patient-facing drape member and a first wing and a second wing on the drape envelope on a distal end. The transport manifold member is disposed within the interior portion. At least a portion of the second, patient-facing drape member is formed with fenestrations. The dressing further includes a bond formed between the first wing and second wing on the drape envelope and a pouch that is formed by the bond. The pouch is for receiving the extremity of the patient. The dressing further includes a sealing device formed on a portion of the pouch for forming a fluid seal with a portion of the patient's skin.

According to another illustrative, non-limiting embodiments, a dressing for forming a fluid seal and delivering reduced pressure to a treatment manifold on a patient's wound site is provided. The dressing includes a transport manifold member, an outer drape member, and an inner drape member. The transport manifold is formed with a receiving site, an extension portion, and an application portion. The outer drape member has a peripheral portion and is sized to have a plan view larger than a plan view of the transport manifold member. Likewise, the inner drape member has a peripheral portion and is sized to have a plan view larger than the plan view of the manifold member and is at least partially fenestrated. The manifold member is disposed between the outer drape member and inner drape member. The dressing further includes an enclosing attachment formed on the periphery of the outer drape member and the periphery of the inner drape member to form a fluid seal between the outer drape member and inner drape member to enclose the manifold member. Further still, the dressing includes a pouch-forming attachment and a sealing device. The pouch-forming attachment is formed on a portion of the outer drape member and inner drape member to form a pouch for receiving the extremity of the patient. The sealing device is coupled to at least a portion of the inner drape member for forming a fluid seal between at least a portion of the inner drape member and a portion of the patient's skin.

According to another illustrative, non-limiting embodiment, a dressing for treating an extremity on a patient includes a drape envelope having an outer drape member, inner drape member, and an interior portion. The interior portion is formed between the outer drape member and the inner drape member. The inner drape member has fenestrations on at least an application portion. The dressing further includes a transport manifold disposed within the interior portion of the drape envelope and a pouch for receiving the extremity. The pouch includes the drape envelope and a pouch-forming attachment.

According to another illustrative, non-limiting embodiment, a dressing for treating an extremity on a patient includes a transport manifold, an outer drape member, and an inner drape member. The transport manifold has a receiving site, an extension portion, and an application portion. The application portion of the transport manifold includes a first wing portion and a second wing portion. The outer drape member has a first wing portion and a second wing portion, and has a proximal end with a fluid aperture formed therein. The inner drape member has a first wing portion and a second wing portion and has fenestrations proximate the first wing portion and the second wing portion. The dressing further includes an enclosing attachment formed on a periphery of the outer drape member and inner drape member to form an interior portion between the outer drape member and inner drape member. The transport manifold is disposed within the interior portion. The dressing further includes a pouch-forming attachment formed at least between the first wing portion and the second wing portion of the inner drape member to form a pouch for receiving the extremity.

According to another illustrative, non-limiting embodiment, a system for treating a wound on an extremity of a patient includes a treatment manifold for disposing proximate the wound, a dressing for use on the wound on the extremity, and a reduced-pressure source. The dressing includes a transport manifold and a drape envelope. The transport manifold includes a receiving site, an extension portion, and an application portion. The drape envelope has an outer drape member and a second, patient-facing drape member, and also has an interior portion between the outer drape member and the second, patient-facing drape member. The drape envelope is formed with a first wing and a second wing on the drape envelope. The transport manifold member is disposed within the interior portion, and at least a portion of the second, patient-facing drape member is formed with fenestrations. The dressing further includes a bond formed between the first wing and the second wing on the drape envelope and includes a pouch formed by the bond. The pouch is for receiving the extremity of the patient. The dressing also has a sealing device formed on a portion of the pouch for forming a fluid seal with a portion of the patient's skin. The reduced-pressure source is fluidly coupled to the transport manifold.

According to another illustrative, non-limiting embodiment, a method of treating a wound on an extremity of a patient includes providing a dressing for treating the extremity of the type previously mentioned, placing the extremity into the pouch, and forming a fluid seal between the pouch and the patient's skin around the wound. The method may further include attaching a portion of the dressing proximate the receiving site of the transport manifold at a location remote from the wound and delivering reduced pressure to the transport manifold.

According to still another illustrative, non-limiting embodiment, a method of manufacturing a dressing for use on an extremity of a patient includes providing a transport manifold having a receiving site, an extension portion, and an application portion, and forming a drape envelope having an outer drape member and a second, patient-facing drape member. The drape envelope is formed to have an interior portion between the outer drape member and the second, patient-facing drape member and a first wing and a second wing on the drape envelope. The method further includes disposing the transport manifold member within the interior portion and forming fenestrations on at least a portion of the second, patient-facing drape member. The method further includes forming a pouch for receiving the extremity of the patient. The step of forming the pouch includes forming a bond between the first wing and second wing on the drape envelope. The method also includes forming a sealing device on a portion of the pouch for forming a fluid seal with a portion of the patient's skin.

According to another illustrative embodiment, a reduced-pressure bridge for delivering reduced pressure to a treatment manifold includes a transport manifold member formed with a receiving site, an extension portion, and application portion. The reduced-pressure bridge further includes an enclosing drape enclosing the transport manifold member. The enclosing drape is formed with an aperture at a proximal end proximate to the receiving site on the transport manifold for the introduction of reduced pressure and at least one aperture at the distal end for egress of reduced pressure. The reduced-pressure bridge has a length (L) and a width (W), and the reduced-pressure bridge has an aspect ratio (L/W) greater than two, three, four, five, six, seven, eight, or more.

Other features and advantages of the illustrative, non-limiting embodiments will become apparent with reference to the drawings and detailed description that follow.

### BRIEF DESCRIPTION OF THE DRAWINGS

A more complete understanding may be obtained by reference to the following Detailed Description when taken in conjunction with the accompanying Drawings, wherein like numerals indicate like elements throughout, and wherein:

FIG. 1 is a schematic, exploded view of an illustrative embodiment of an occlusive wrapping;

FIG. 2 is a schematic, orthogonal front view of the illustrative embodiment of the occlusive wrapping of FIG. 1 shown with a portion broken away;

FIG. 3 is a schematic, plan view of an illustrative embodiment of a fluid manifold shown in a flat position;

FIG. 4 is a schematic, perspective view of an illustrative embodiment of a contoured porous pad;

FIG. 5 is a schematic, top plan view of the contoured porous pad of FIG. 4;

FIGS. 6A-6G are schematic, perspective views of the negative pressure wound treatment dressing system, illustrating the steps of applying the dressing system to a wound on the heel of a foot;

FIG. 7 is a schematic, side view of an illustrative embodiment of the occlusive wrapping;

FIG. 8 is a schematic, perspective of an illustrative embodiment of a system for treating an extremity shown fully deployed on an extremity;

FIG. 9 is a schematic, plan view of a dressing for deploying on an extremity for use with the system of FIG. 8 shown in a flat, partially assembled state;

FIG. 10 is a schematic, exploded, perspective view of the dressing of FIG. 9;

FIG. 11 is a schematic, perspective view of the dressing of FIGS. 9-10 shown in an assembled state;

FIG. 12 is a schematic, cross sectional view along line 12-12 of FIG. 10;

FIG. 13 is a schematic, elevation view of a portion of the dressing of FIGS. 8-11 showing a distal end of a pouch;

FIG. 14 is a schematic, perspective view of a healthcare provider sizing a treatment manifold for deployment in a wound bed;

FIG. 15 is a schematic, perspective view of the sized treatment manifold of FIG. 14 shown sized for the wound bed;

FIG. 16 is a schematic, perspective view of the treatment manifold of FIGS. 14-15 shown deployed in a wound bed of a patient;

FIG. 17 is a schematic, perspective view of a dressing for use on an extremity shown being initially deployed on a heel of the patient;

FIG. 18 is a schematic, perspective view of the dressing of FIG. 17 shown with the healthcare provider removing a first release liner;

FIG. 19 is a schematic, perspective view of the dressing of FIG. 17 shown with the healthcare provider removing a second release liner;

FIG. 20 is a schematic, perspective view of the dressing of FIG. 17 shown with the healthcare provider using a finger hold to begin to remove a portion of a protective liner;

FIG. 21 is a schematic, perspective view of the dressing of FIG. 17 shown with the healthcare provider using another finger hold to begin to remove a portion of a protective liner;

FIG. 22 is a schematic, perspective view of the dressing of FIG. 17 shown with the healthcare provider removing a receiving site release liner;

FIG. 23 is a schematic, perspective view of the dressing of FIG. 17 shown with the healthcare provider deploying a portion of the dressing proximate a receiving site;

FIG. 24 is a schematic, perspective view of the dressing of FIG. 17 shown with the healthcare provider beginning to deploy a reduced pressure interface on to the dressing;

FIG. 25 is a schematic, perspective view of the dressing of FIG. 17 shown with the healthcare provider removing the reduced-pressure interface liner; and

FIG. 26 is a schematic, perspective of another illustrative embodiment of a system for treating an extremity shown fully deployed on an extremity.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined only by the appended claims.

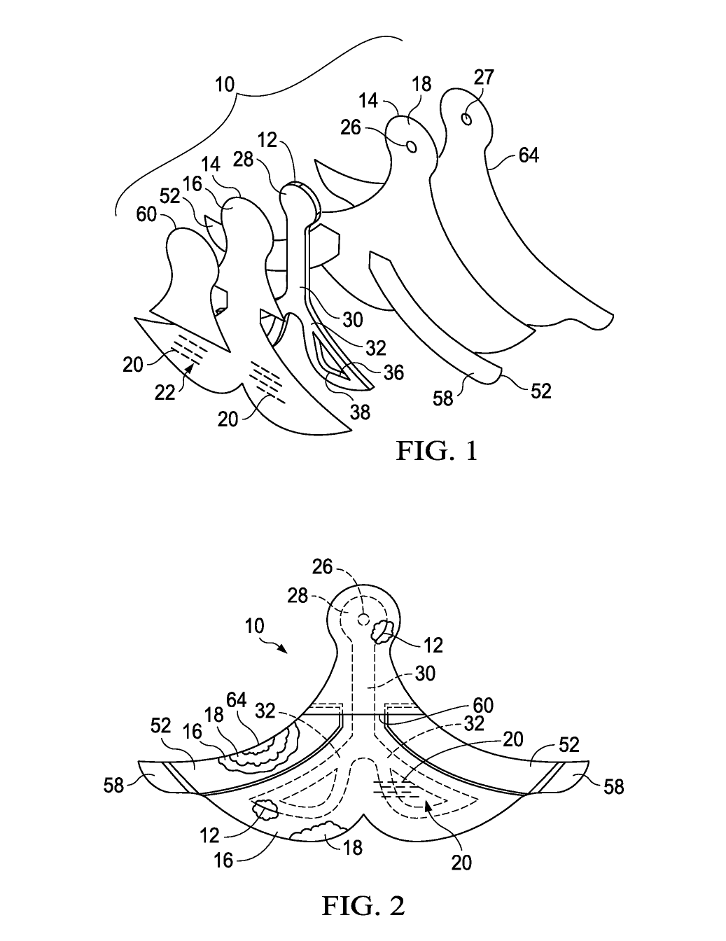

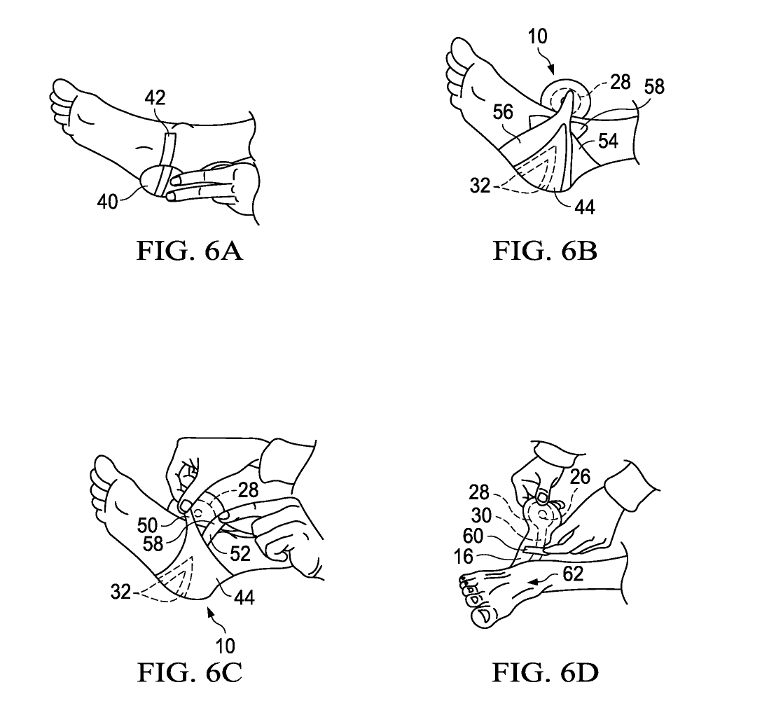

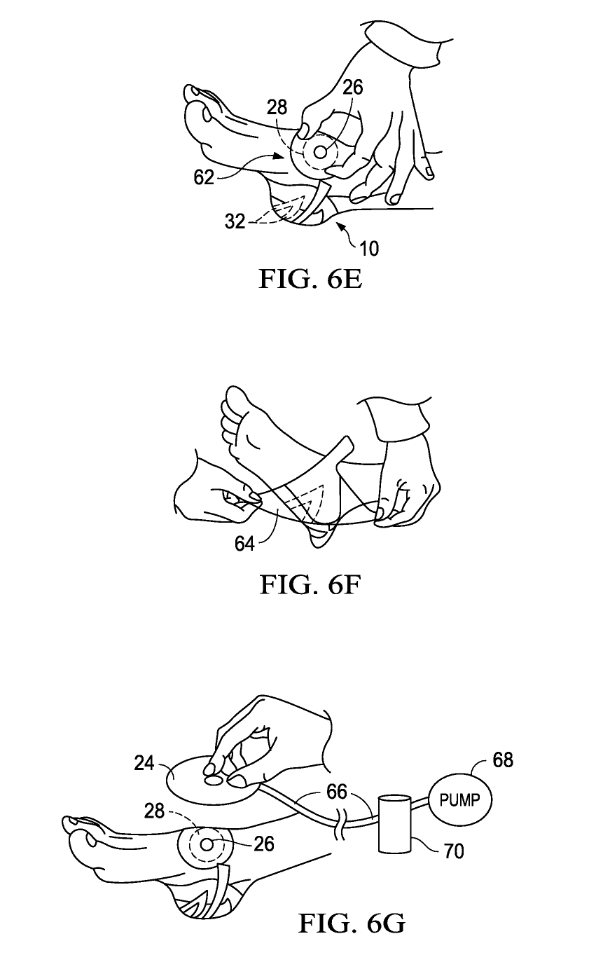

Referring primarily to FIGS. 1-3, an illustrative, non-limiting embodiment of an occlusive wrapping 10 for use as part of a negative pressure dressing system is presented. It should be apparent that “occlusive” means fluid impermeable once the occlusive wrapping 10 is fully deployed on a patient and also includes reduced pressure being delivered to the occlusive wrapping 10. The occlusive wrapping 10 includes a fluid manifold 12 enclosed within a drape or occlusive drape 14. The occlusive drape 14 is preferably made of a clear vapor permeable polyurethane material. The drape 14 includes a wound facing layer or drape member 16 and an outer layer or drape member 18 that are sealed along their periphery to secure the fluid manifold 12 within the wound facing layer 16 and the outer layer 18 of the drape 14. Multiple fenestrations 20 are formed along a wound contact region 22 of the wound facing layer 16, so as to allow for fluid communication through the fluid manifold 12. The fluid manifold 12 allows for attachment of a fluid communication port connector 24 (FIG. 6G), which interfaces with or is part of a fluid port or aperture 26 in the outer layer 18, at a position away from the wound site. Although the protective liner 64 is typically removed before applying a reduced pressure interface, the protective liner 64 may have an aperture 27.

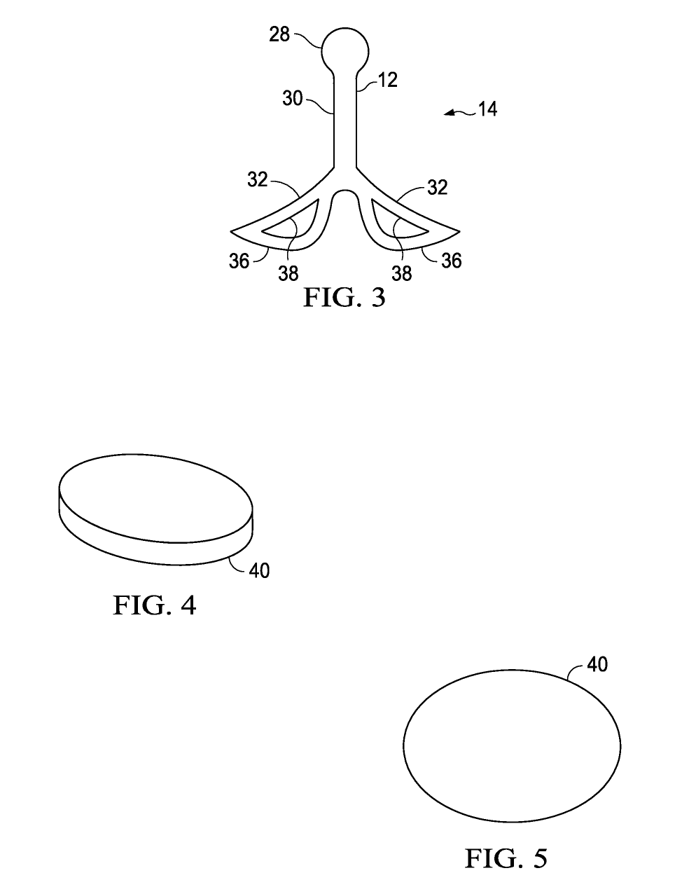

Coupling the fluid communicator port connector 24 at a position away from the wound site is desirable. Indeed, the type of wounds desired to be treated by illustrative embodiments herein are often situated at positions that have at times been difficult to maintain a reduced pressure using traditional negative pressure wound therapy techniques. The occlusive wrapping 10 of the present invention overcomes this difficulty by communicating the negative pressure along the fluid manifold 12 from a remote site to a wound site while maintaining a low profile. The fluid manifold 12 of the occlusive wrapping 10 is illustrated in FIG. 3.

FIG. 3 presents the fluid manifold 12 of the occlusive wrapping 10 in a flat position. The fluid communication port connector 24 will be attached proximate a receiving site 28. The receiving site 28 is preferably a circular shaped foam section and is approximately the size of the fluid communication port connector 24, but may be bigger or smaller in other embodiments. An extension region or portion 30 extends between the receiving site 28 and fluid communicator arms 32 and more particularly to wound contact regions 22. The fluid communicator arms 32 may terminate in loops 36, having openings 38 for viewing the wound perimeter. In an alternative embodiment, fluid communicator fingers extend distally from the fluid communicator arms 32.

An illustrative embodiment of a pad or contoured pad 40 is presented in FIGS. 4 and 5. The contoured pad 40 is utilized as a wound contact screen to prevent unwanted adhesions, direct fluid away from the wound through pores within the pad 40, and exert contracting forces on the wound margins as negative pressure is applied through the manifold 12 and to the contoured pad 40. The contoured pad 40 and fluid manifold 12 of the occlusive wrapping 10 are preferably made of a porous polymer foam material, such as polyurethane or polyvinyl alcohol foam. The contoured pad 40 is preferably constructed into an elliptical shape as illustrated clearly in FIG. 5, but may also be more symmetrically circular to accommodate more uniform wounds. The contoured pad 40 may be concave or take on other shapes.

In practice, the contoured pad 40 is trimmed to the area dimensions of the wound and is placed in the wound, which may be on the heel of a foot, and between the wound and the fluid manifold 12, as illustrated in FIGS. 6A-6B. The contoured pad 40 may be secured to the wound site by an adhesive strip 42, as shown in FIG. 6A, or by other means known in the art. In one embodiment, the adhesive strip 42 is constructed of the same vapor permeable polyurethane material as the occlusive drape 14, having an adhesive coated on one side for securing the strip to the pad 40 and the intact skin surrounding the wound.

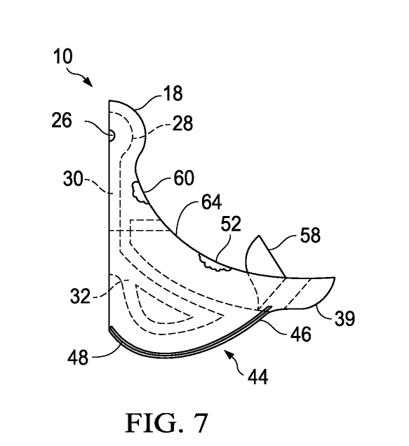

The occlusive wrapping 10 forms a pouch 44 when it is folded along its centerline and the lower edges 46 are bonded together at bond site 48 near a lower edge 46 as shown in FIG. 7. In practice, the pouch 44 is placed over the wound and contoured pad 40, as illustrated in FIGS. 6B and 6C. Fingerholds 50 are provided to facilitate grasping the pouch 44 and holding the pouch 44 in place as liners 52 are removed to expose adhesive or adhesive backing on layer 16. The edges 54, 56 of the pouch 44 are brought into contact with the intact skin surrounding the wound by means of an adhesive backing The removable liner 52 is pulled off, starting at finger tab 58, exposing the adhesive, as shown in FIG. 6C. The removable liner 52 may be removably adhered to the adhesive backing to protect the adhesive during handling.

As illustrated in FIG. 6D, a second removable liner 60 is adhered to the wound facing layer 16 proximate the receiving site 28 to protect the adhesive on the wound facing layer 16 along the receiving site 28 during handling. The second removable liner 60 is removed prior to adhesion of the wound facing layer 16 proximate the receiving site 28 to a region 62 of intact skin away from the wound site, so as to prevent dislodgement of the fluid communication port connector 24 during movement of the patient, as shown in FIG. 6E. A removable, protective liner 64 may be overlaid on the occlusive drape 14, so as to provide rigidity to the manifold during handling, and which is removed after securing the pouch 44 to the wound site, as shown in FIG. 6F.

Referring to FIG. 6G, the fluid communication port connector 24 is positioned over the receiving site 28, and is secured in place also by means of an adhesive contact surface on the communication port connector 24. As known in the art of negative pressure wound therapy, a fluid communication means 66, such as a flexible silicone tube or conduit, communicates negative pressure to the wound from a negative pressure source 68, such as a portable pump. A collection canister 70 is positioned in line between the negative pressure source 68 and the fluid communication means 66 to collect any exudates that may be drawn from the wound.

Referring to FIGS. 1-7, according to an illustrative embodiment, a wound treatment dressing for applying negative pressure wound therapy to a wound of an extremity, such as on the heel of a foot, is presented. The dressing includes the fluid manifold 12 positioned within the drape 14, which has the wound facing layer 16 and the outer layer 18, to form the contoured occlusive wrapping 10 for placement over a wound on an extremity. The contoured porous pad 40 is placed adjacent or within the wound, such that the contoured pad 40 is enveloped by the occlusive wrapping 10. The occlusive wrapping 10 forms a fluid seal over the contoured porous pad 40, which is within or on the wound. The flexible tubing 66 is attached to or through a fluid communication port connector 24, which is cooperates with the fluid aperture 26 in the outer layer 18 of the occlusive wrapping 10, so as to allow for fluid communication of negative pressure to the contoured pad 40 from a source of negative pressure 68 connected to an opposite end of the flexible tubing 66. The negative gauge pressure is communicated from the source 68, through the tube 66, through the fenestrations 20 of the occlusive wrapping 10, such that negative gauge pressure is applied to the wound on the extremity. The fluid manifold 12 serves to direct the negative pressure from a position away from the wound site to the contoured pad 40 at the wound site.

According to an illustrative embodiment, an apparatus includes a contoured porous pad 40 positioned within a fenestrated and contoured occlusive wrapping, e.g., occlusive wrapping 10, for placement over a wound on an extremity, such as the heel of a human foot. The contoured pad 40 is placed adjacent or within the wound, such that the contoured pad 40 is enveloped by the occlusive wrapping 10. Flexible tubing 66 is attached to or through an opening 26 of the drape 14, so as to allow for fluid communication of negative pressure to the contoured pad 40 from a source of negative pressure 68 connected to an opposite end of the flexible tubing 66. The negative gauge pressure is communicated from the source 68, through the tube 66, through the fluid manifold 12, through the fenestrations 20 of the occlusive wrapping 10, such that negative gauge pressure is applied to the wound on the extremity. The occlusive wrapping 10 serves to manifold the negative pressure from a position away from the wound site to the pad 10 at the wound site. Such an arrangement allows for the tubing 66 to connect to the occlusive wrapping 10 at a position least likely to be dislodged during normal ambulation of the patient.

Attachments means are formed on the outer edges of the occlusive wrapping 10 to form an airtight seal over the wound. Such attachment means may be an adhesive coated on the occlusive wrapping 10 for attachment to an intact area of tissue or skin. The adhesive is coated on the perimeter edge of the occlusive wrapping 10 such that a fluid seal is formed when the occlusive wrapping 10 is secured over the concave pad.

The collection canister 70 is provided between the negative pressure source 68 and the occlusive drape 10 for collecting any effluents that may be drawn from the wound during application of negative pressure.

The tubing 66 is connected to the drape 14 and pad 40 through an opening 26 in the drape 14, or through a flanged tubing connector as has been described in U.S. Pat. No. 6,345,623, entitled “Surgical drape and suction head for wound treatment,” to Heaton, et al., which is incorporated herein by reference as though fully set forth. Alternative embodiments allow the tubing 66 to be placed directly within or embedded in the contoured pad 40.

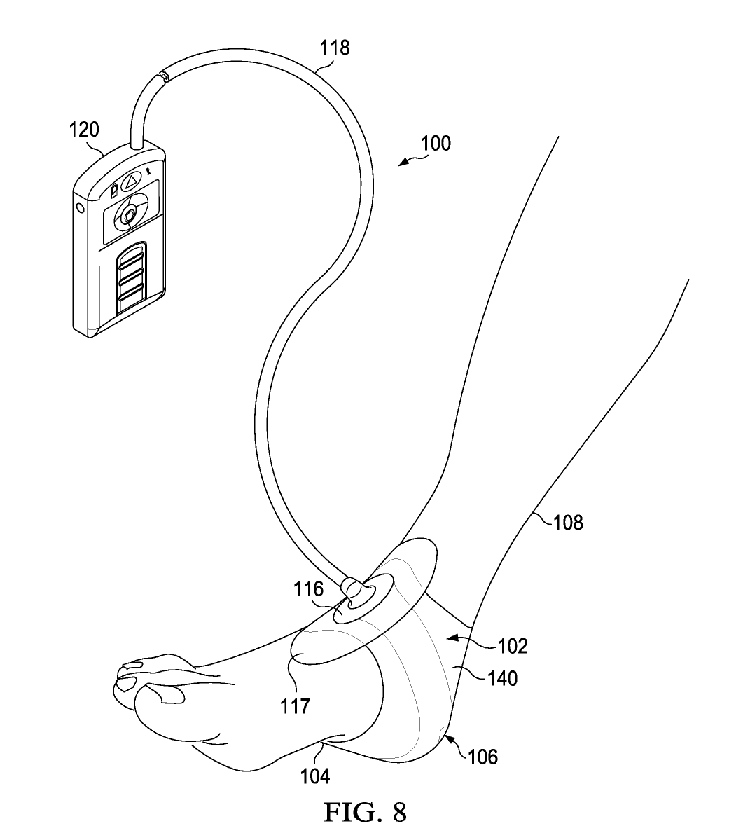

Referring now to FIGS. 8-25, an illustrative, non-limiting embodiment of a reduced pressure treatment system 100 is presented that includes a dressing 102 for treating an extremity 104, such as a heel 106, of a patient 108 with reduced pressure, or negative pressure. Typically a wound 110 having a wound bed 112 is located on the extremity 104. A treatment manifold 114, or pad, is placed in the wound bed 112 and the dressing 102 is used to form a fluid seal over the treatment manifold 114 and provides a location remote from the wound 110 where a reduced pressure interface 116, or reduced pressure port connector, may be attached. A reduced pressure delivery conduit 118 fluidly couples the reduced pressure interface 116 with a reduced pressure source 120.

The term “manifold” as used herein generally refers to a substance or structure that is provided to assist in applying reduced pressure to, delivering fluids to, or removing fluids from a tissue site, e.g., wound 110 or another location. The treatment manifold 114 typically includes a plurality of flow channels or pathways that distribute fluids provided to and removed from the wound 104 near the treatment manifold 114. In one illustrative embodiment, the flow channels or pathways are interconnected to improve distribution of fluids provided or removed from the wound 104. The treatment manifold 114 may be a biocompatible material that is capable of being placed in contact with the wound 104 and distributing reduced pressure to the wound 104. Examples of treatment manifolds 114 may include, for example, without limitation, devices that have structural elements arranged to form flow channels, such as, for example, cellular foam, open-cell foam, porous tissue collections, liquids, gels, and foams that include, or cure to include, flow channels. The treatment manifold 114 may be porous and may be made from foam, gauze, felted mat, or any other material suited to a particular biological application.

In one embodiment, the treatment manifold 114 is a porous foam and includes a plurality of interconnected cells or pores that act as flow channels. The porous foam may be a polyurethane, open-cell, reticulated foam, such as GranuFoam® material manufactured by Kinetic Concepts, Incorporated of San Antonio, Tex. Other embodiments may include “closed cells.” These closed-cell portions of the manifold may contain a plurality of cells, the majority of which are not fluidly connected to adjacent cells. The closed cells may be selectively disposed in the treatment manifold 114 to prevent transmission of fluids through perimeter surfaces of the treatment manifold 114. In some situations, the treatment manifold 114 may also be used to distribute fluids such as medications, antibacterials, growth factors, and various solutions to the wound 104. Other layers may be included in or on the treatment manifold 114, such as absorptive materials, wicking materials, hydrophobic materials, and hydrophilic materials. The treatment manifold 114 may be contoured to fit an extremity 104, such as the heel 106.

The reduced-pressure source 120 provides reduced pressure. The reduced-pressure source 120 may be any device for supplying a reduced pressure, such as a vacuum pump, wall suction, or other source. While the amount and nature of reduced pressure applied to a tissue site will vary according to the application, the reduced pressure will typically be between −5 mm Hg and−500 mm Hg and more typically between −100 mm Hg and −300 mm Hg.

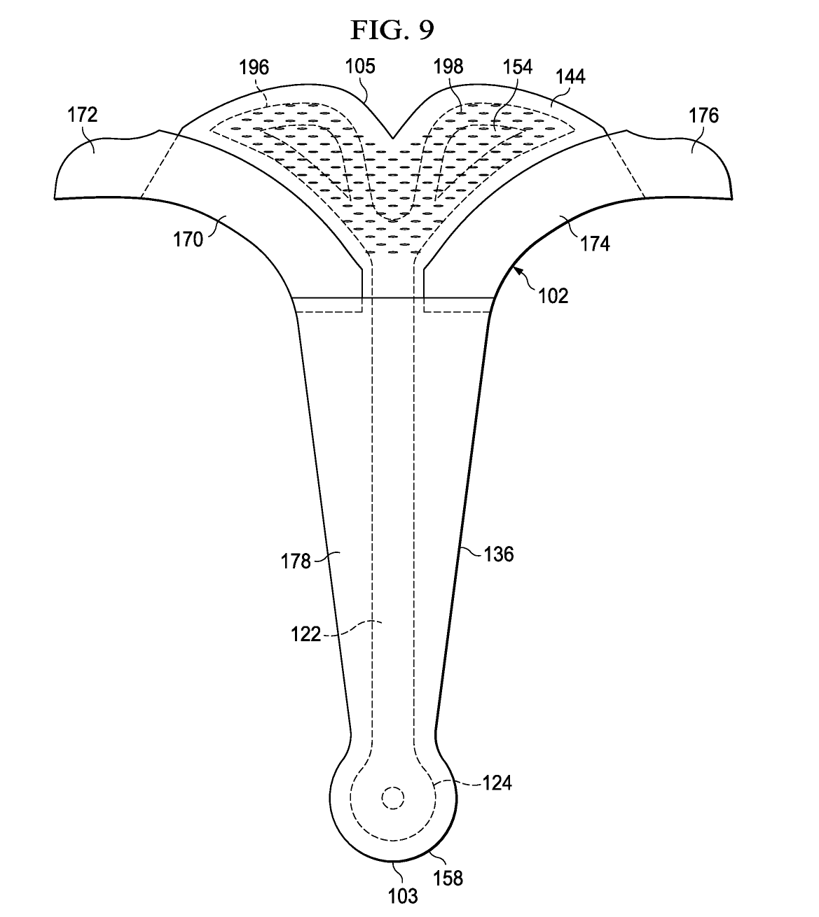

Referring now primarily to FIGS. 9-13, the dressing 102 has a distal end 105 and a proximal end 103. The dressing 102 may include a transport manifold 122 having a receiving site 124, an extension portion 126, and an application portion 128. The transport manifold 122 may be formed from the same material as the treatment manifold 114 or any of the materials mentioned in connection with the treatment manifold 114. The transport manifold 122 may be formed with a first wing portion 130 and a second wing portion 132 formed on a distal end 134. The first wing portion 130 may have an opening 131 and the second wing portion 132 may have a second opening 133. The extension portion 126 may extend from the receiving site 124 to the start of the first wing portion 130 and the second wing portion 132. The extension portion 126 may have an aspect ratio (longer dimension/shorter dimension in plan view like in FIG. 3) greater than two, greater than three, greater than four, or even greater still.

The transport manifold 122 is enclosed within a drape envelope 136 that is formed with a first wall 138, or an outer drape member 140, and a second, patient-facing wall 142, or inner drape member 144. The drape envelope 136 forms an integrated drape and makes application easy for the healthcare provider 206. The outer drape member 140 has a first peripheral portion 146 and the inner drape member 144 has a second peripheral portion 148. The outer drape member 140 is sized to have a larger surface area than the surface of the transport manifold 122. In other words, the outer drape member 140 is sized to have a plan view larger than the plan view of the transport manifold member 122. Similarly, the inner drape 144 is sized to have a surface area larger than the surface area of the transport manifold 122.

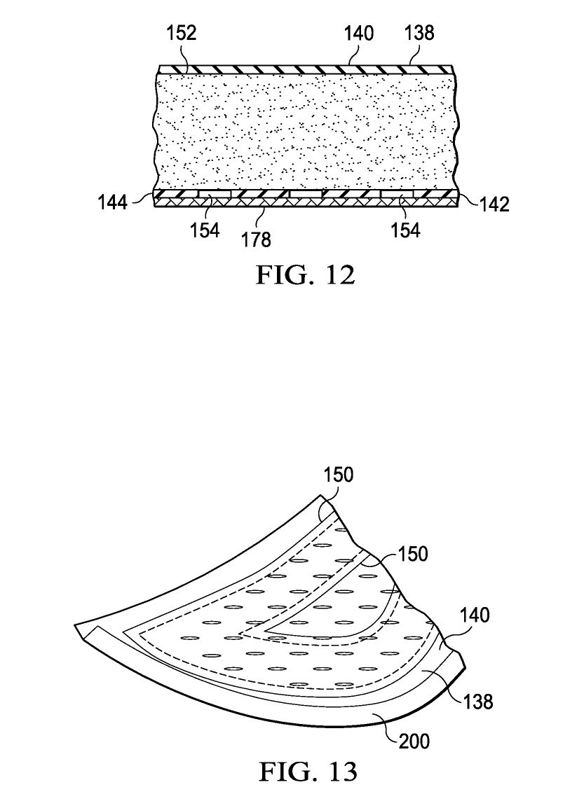

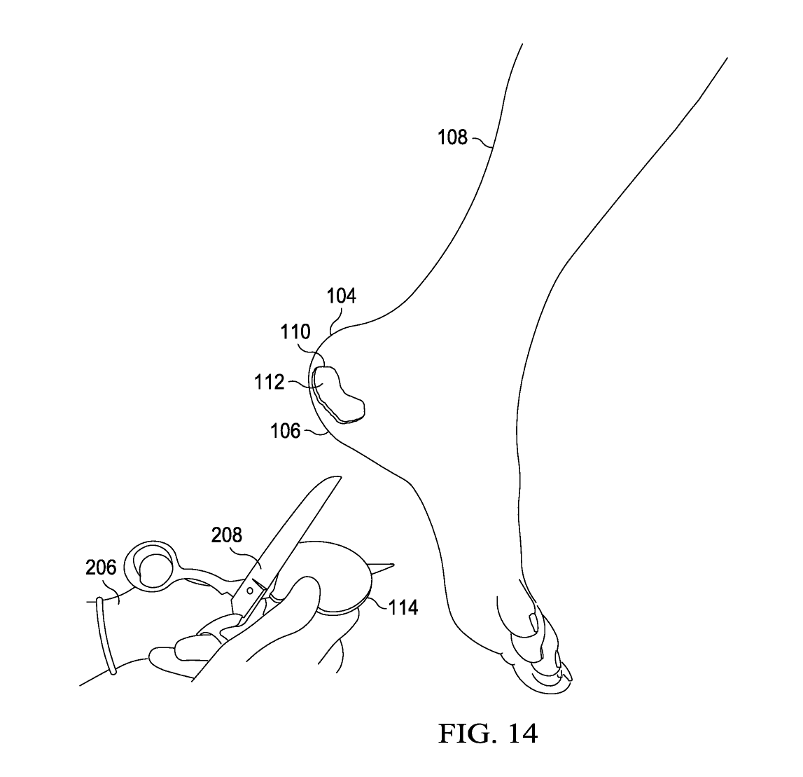

An enclosing attachment or connection 150, shown clearly in FIG. 13, may be formed anywhere between the peripheral portions 146, 148 and just outboard of the transport manifold member 122. In addition, the enclosing attachment 150 may also be formed on an inboard portion of the fluid manifold 112 in the openings 131, 133. The enclosing attachment 150 forms the interior portion 152 of the drape envelope 136 and holds and substantially seals the transport manifold member 122 in the interior portion 152. One apparent exception to the seal is that the drape envelope 136 includes limited apertures, e.g., fenestrations 154, aperture 156, etc. A cross section of a portion of the dressing 102 is shown in FIG. 12 and the interior portion 152 may be seen. The enclosing attachment 150 formed on the outer drape member 140 and inner drape member 144 forms a fluid seal between the drape members 140, 144 and encloses the transport manifold 122. The second, patient-facing wall 142, or inner drape member 144, is formed with the fenestrations 154 at least on a portion and typically at least on an application portion 155, or wound contact region. The fenestrations 154 may be any apertures suitable for allowing fluids to flow into the interior portion 152 of the drape envelope 136. In addition, the fluid aperture 156 may be formed on a proximal end 158 of the dressing 102 for interfacing with the reduced-pressure interface 116.

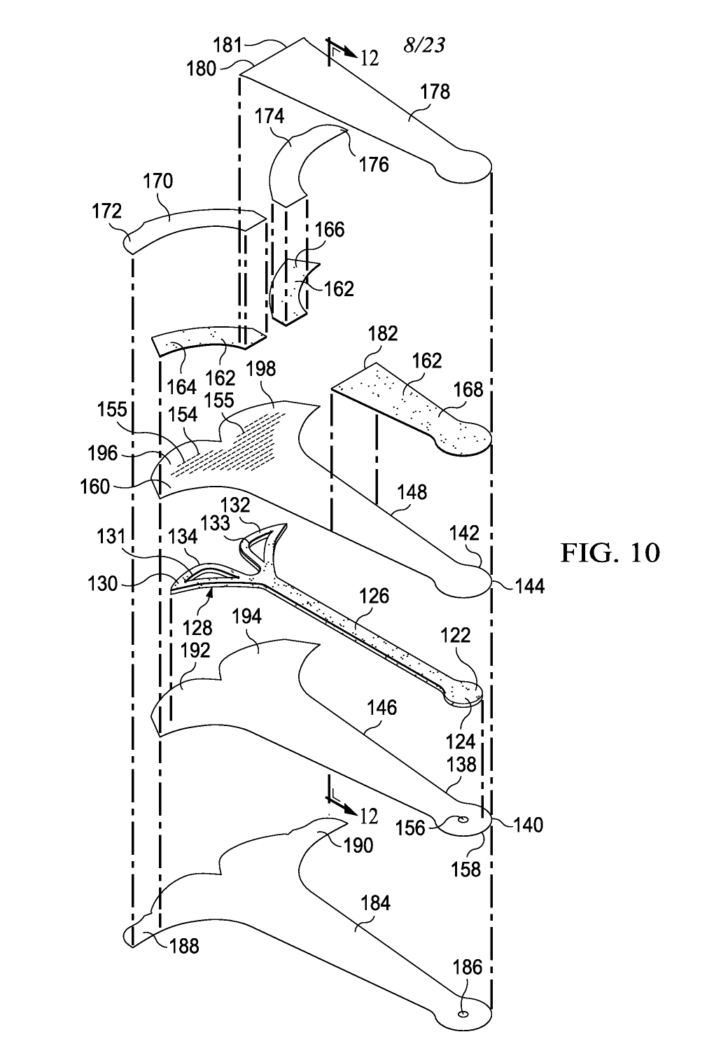

An exterior 160 of the inner drape member 144 or at least a portion of the exterior 160 has an adhesive 162 applied. The adhesive 162 may be any material suitable for assisting or helping to form a fluid seal between the inner drape member 144 and a portion of the patient's skin. The adhesive 162 may include a contact adhesive, hydrocolloid, or any device for removably holding surfaces. As shown clearly in FIG. 10, the adhesive 162 may include a first wing adhesive 164, a second wing adhesive 166, and a receiving site adhesive 168.

The adhesive 162 or portions of the adhesive 162 may be covered by one or more release liners prior to use. For example, a first release liner 170 may be releasably coupled to first wing adhesive 164. The first release liner 170 may include a finger hold portion 172 that extends beyond the first wing adhesive 164 and facilitates removal of the release liner 170. Similarly, a second release liner 174 may be releasably coupled to the second wing adhesive 166. The second release liner 174 includes a second finger hold 176 that extends beyond the second wing adhesive 166 and facilitates removal of the second release liner 174. A receiving site release liner 178 is releasably coupled to the receiving site adhesive 168. The receiving site release liner 178 may have a distal portion 180 that extends beyond a distal portion 182 of the receiving site adhesive 168 and thereby forms a finger hold 181 for removing the receiving site release liner 178. The distal portion 180 of the receiving site release liner 178 may rest in the un-deployed state on a portion of the first release liner 170 and the second release liner 174.

A protective liner 184 may be applied to an exterior surface of the outer drape member 140 to provide stability and increased rigidity and to facilitate deployment of the dressing 102. The protective liner 184 may be held to the exterior of the outer drape member 140 by static charge between the materials or with a mild adhesive or other device. The protective liner 184 may include a second fluid aperture 186 and may include a first finger hold 188 and a second finger hold 190 that facilitate removal of the protective liner 184 from the outer drape member 140.

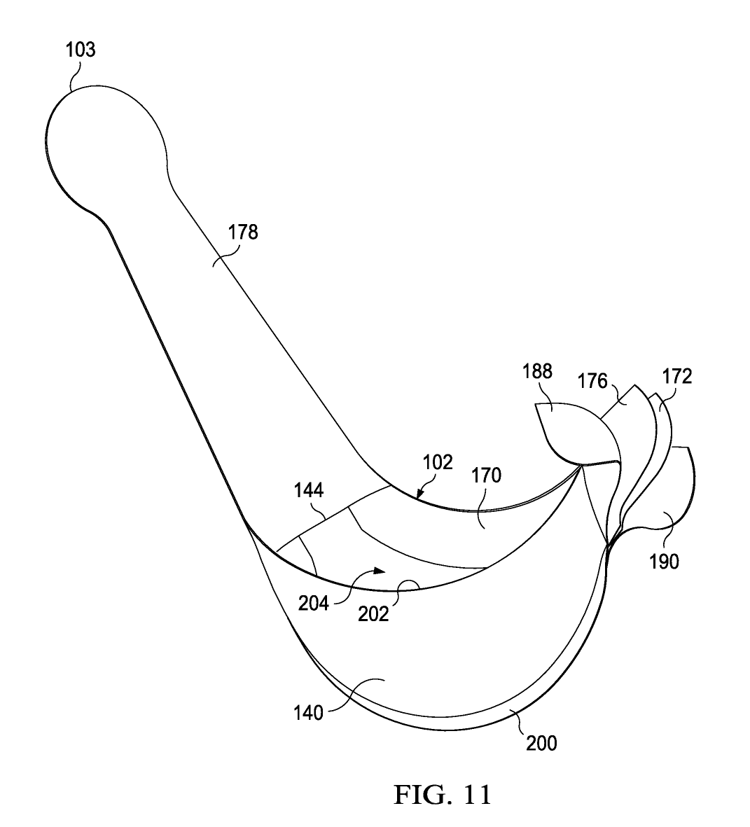

The outer drape member 140 may be formed with a first wing portion 192 and a second wing portion 194 that substantially correspond, although a little larger, with the first wing portion 130 and second wing portion 132 of the transport manifold 122. Likewise, the inner drape member 144 may be formed with a first wing portion 196 and a second wing portion 198 that substantially correspond, although a little larger, with the first wing portion 130 and second wing portion 132 of the transport manifold 122. As shown clearly in FIG. 11, a pouch-forming attachment 200 may be formed or applied to the drape envelope 136 to form a pouch 202 having a pouch interior 204. The pouch-forming attachment 200 may couple the first wing portion 192 and second wing portion 194 of the outer drape member 140 with the first wing portion 196 and second wing portion 198 of the inner drape member 144 to form the pouch 204. The pouch 202, and more particularly the pouch interior 204, is for receiving the patient's extremity 104, such as the heel 106.

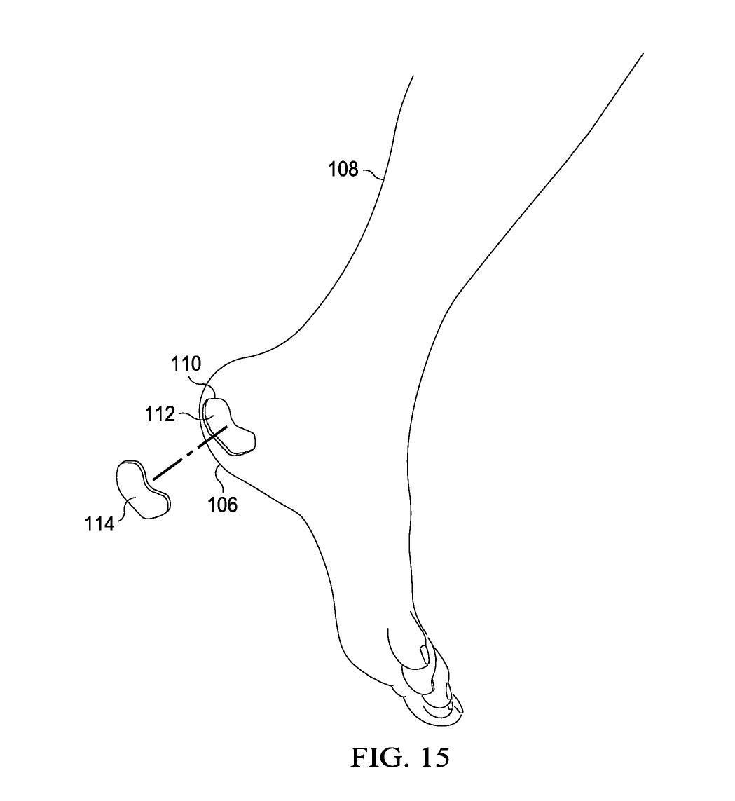

Referring now to FIGS. 14-25, the application of dressing 102 as part of a reduced pressure treatment system 100 will now be presented. Referring initially to FIGS. 14 and 15, the healthcare provider 206 may use an implement 208 to cut the treatment manifold 114 to an appropriate size to fit within the wound bed 112 of the wound 110. In other embodiments, the treatment manifold 114 may have perforations or other devices for allowing the healthcare provider 206 to size the treatment manifold 114 without requiring an implement 208.

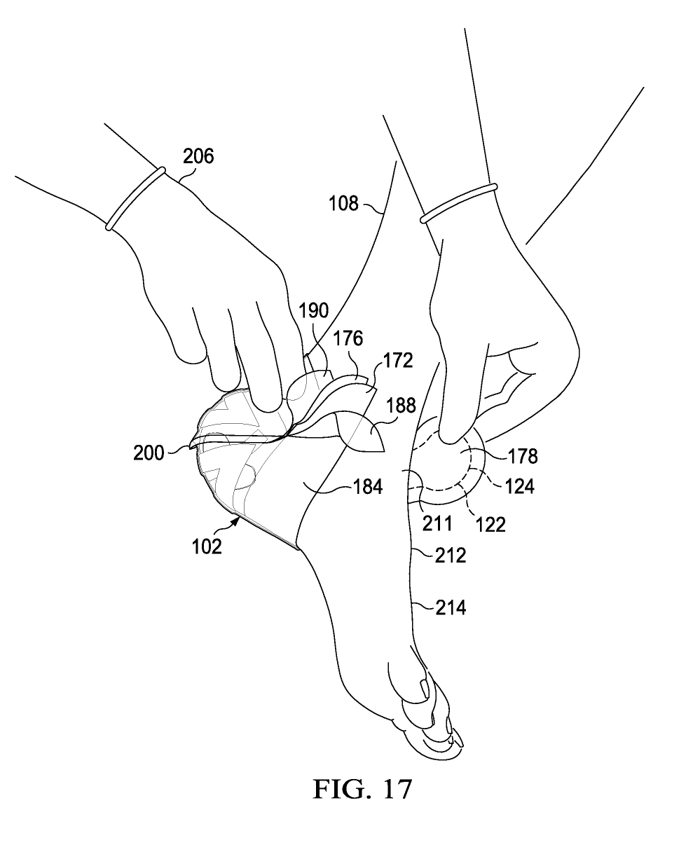

Referring now to FIG. 17, the treatment manifold 114 may be initially held or secured in the wound bed 112 using an adhesive drape strip 210. In the embodiment shown, the adhesive drape strip 210 is transparent and the treatment manifold 114 is visible through the adhesive drape strip 210.

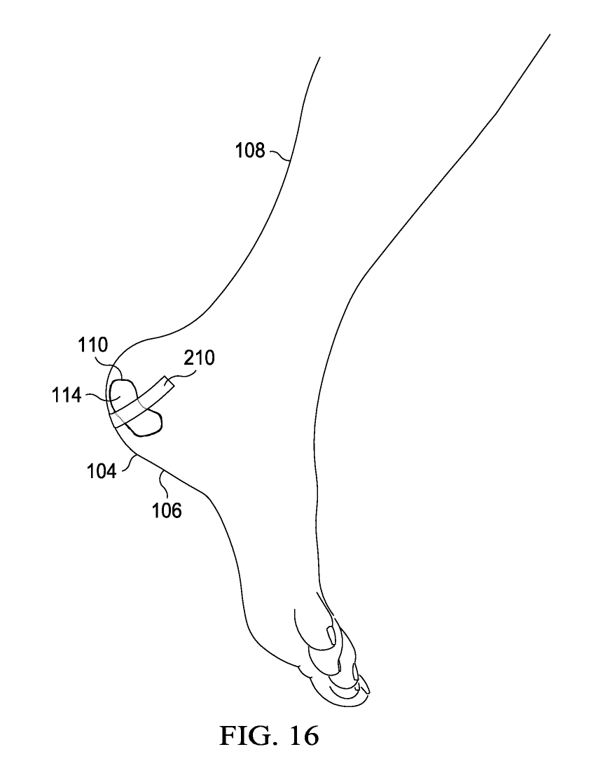

Referring now to FIG. 16, the healthcare provider 206 places the extremity 104 into the pouch interior 204 (FIG. 11) and positions the dressing 102 so that the receiving site 124 is located at a remote location 211, such as a dorsal portion 212 of the patient's foot 214. In one embodiment, the dressing 102 is made with protective liner 184, outer drape member 140, and inner drape member 144 that are transparent, and accordingly additional features may be seen in FIG. 17. With the exception of the first finger hold 172 and the second finger hold 176, most of the exterior portion of the dressing 102 shown at this point of its application is the protective liner 184.

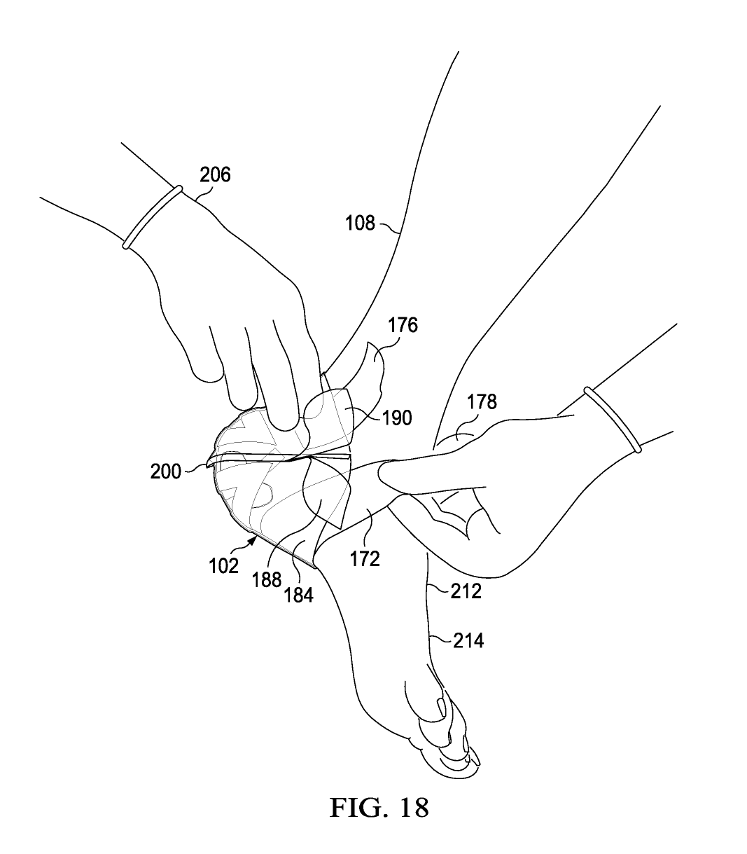

Referring now to FIG. 18, once the dressing 102 is in the desired position, the healthcare provider 206 pulls on the first finger hold 172 to remove the first release liner 170 thereby exposing first wing adhesive 164 to the patient's skin. The healthcare provider 206 smoothes out the first wing 196 of the dressing 102 to from a fluid seal.

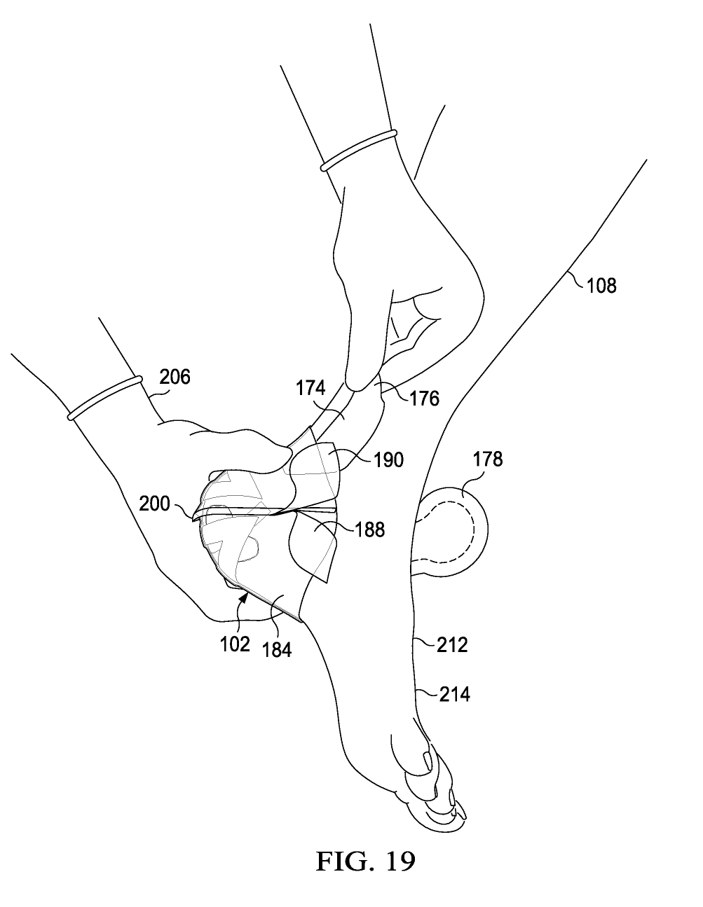

Referring now to FIG. 19, the healthcare provider 206 also pulls on the second finger hold 176 to remove the second release liner 174 to expose the second wing adhesive 166 to the patient's skin. The healthcare provider 206 smoothes out the second wing 198 to form a fluid seal with the patient's skin. The first wing adhesive 164 and second wing adhesive 166 substantially seal the pouch 202 around the wound 110 except for possibly one portion that will be sealed by the receiving site adhesive 168 when the receiving site release liner 178 is removed. The sealed pouch 202 holds the extremity and inhibits leaks—even during movement of the extremity 104.

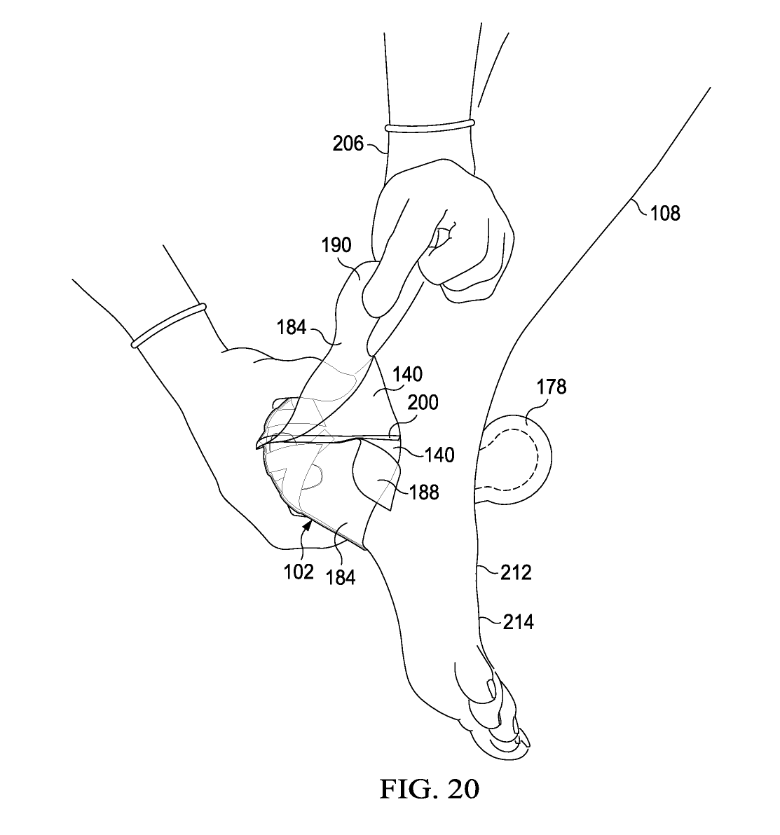

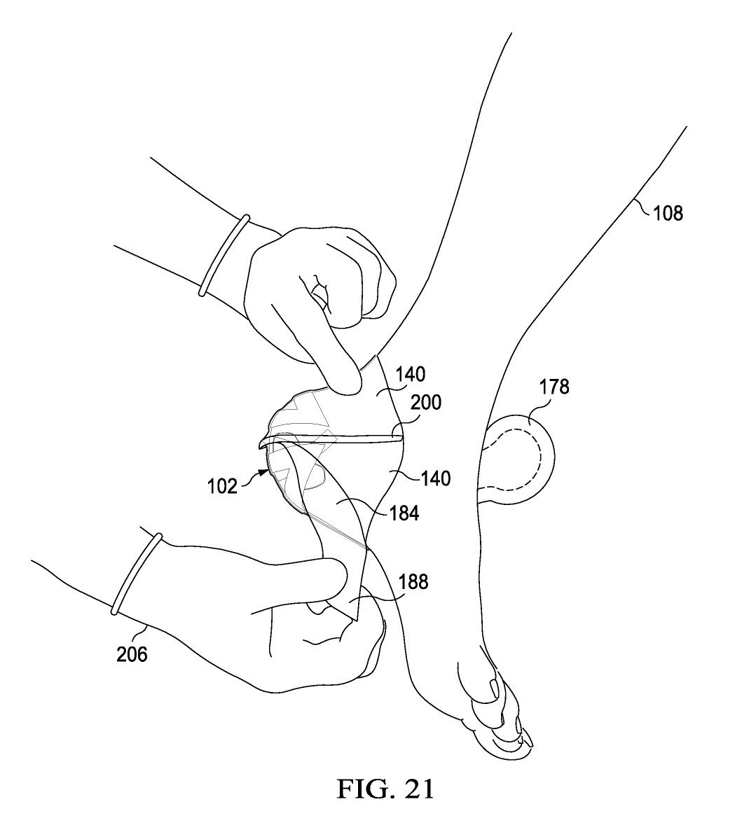

Referring now to FIG. 20, the healthcare provider 206 may then pull on the second finger hold 190 of the protective liner 184 to remove the protective liner 184 from the outer drape member 140. Similarly, referring to FIG. 21, the healthcare provider 206 pulls the first finger hold 188 of the protective liner 184 to finish removing the protective liner 184. It should be noted that the protective liner 184 may be one, two, or more pieces.

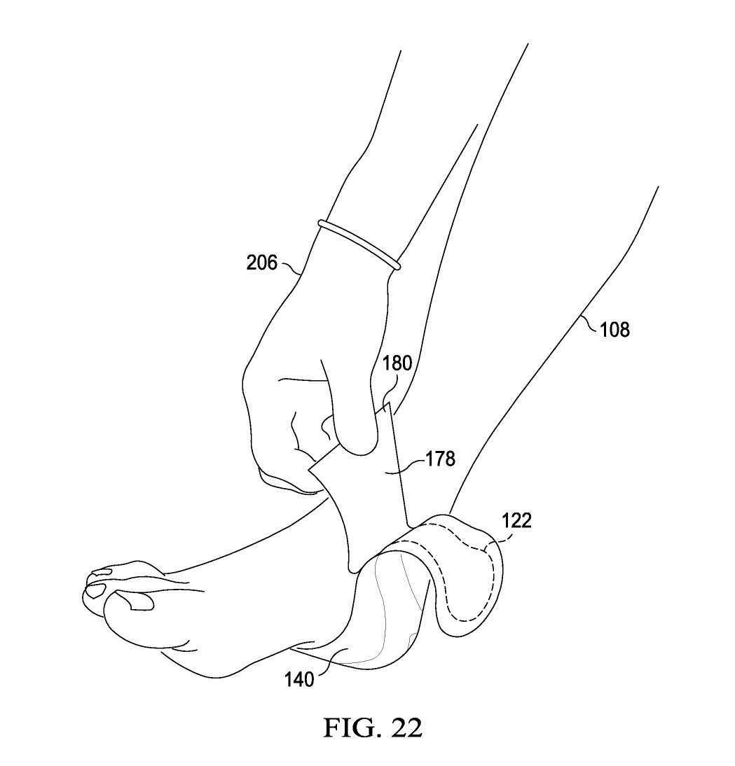

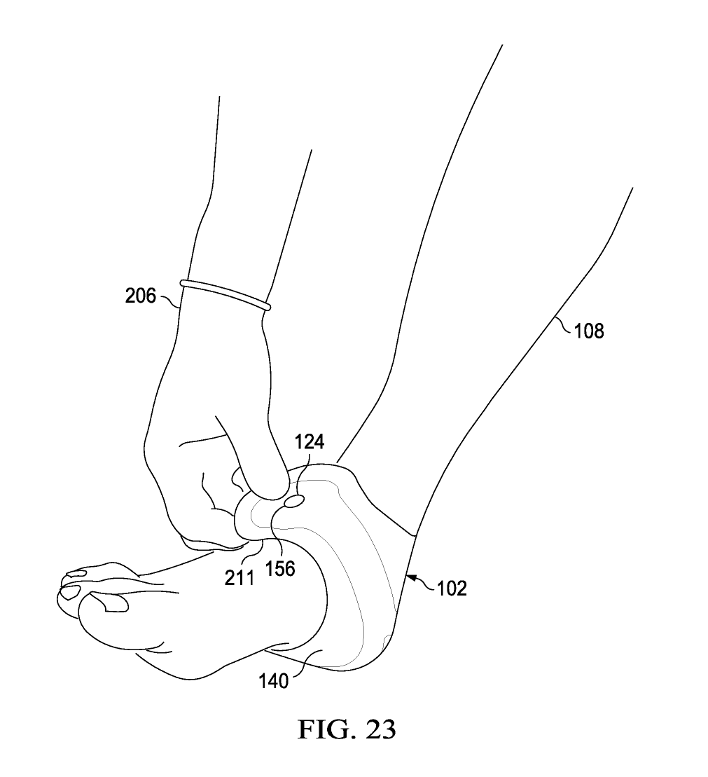

Referring now to FIG. 22, the healthcare provider 206 then may pull the distal portion 180 of the receiving site release liner 178 and remove the receiving site release liner 178. Removal of the receiving site release liner 178 exposes the receiving site adhesive 168 and completes forming a fluid seal around the wound 110 if a sufficient seal had not already been formed. The healthcare provider 206 smoothes down the drape envelope 136 over the receiving site adhesive 168 to form a good fluid seal. Referring to FIG. 23, it can be seen that the healthcare provider 206 places the receiving site 124 at the desired remote location 211. At this point, the deployment of the dressing 102 is complete. The ability to apply the reduced pressure interface 116 at the remote location 211 provides greater comfort for the patient 108 and allows for faster dressing changes.

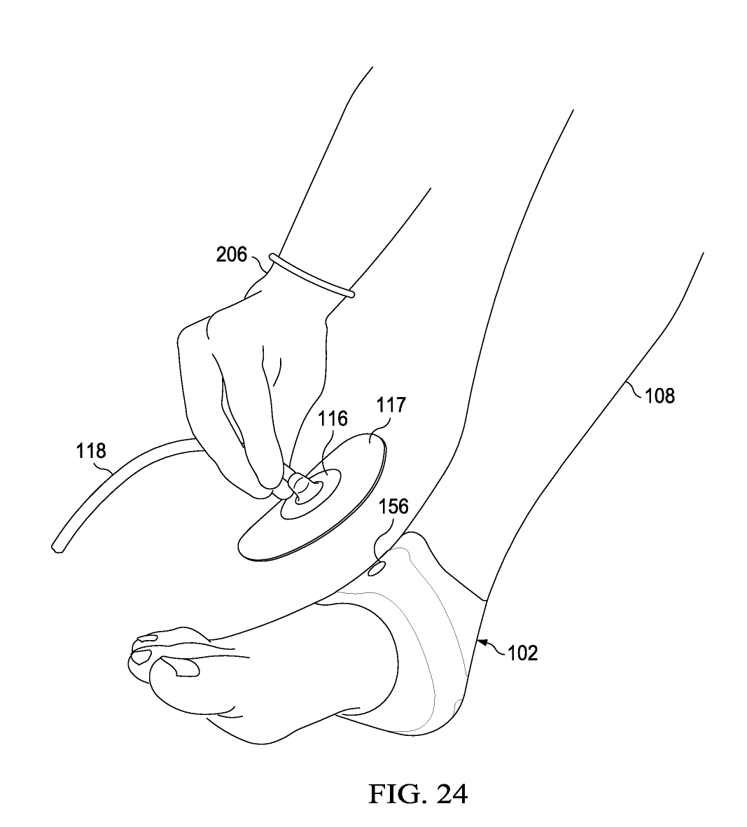

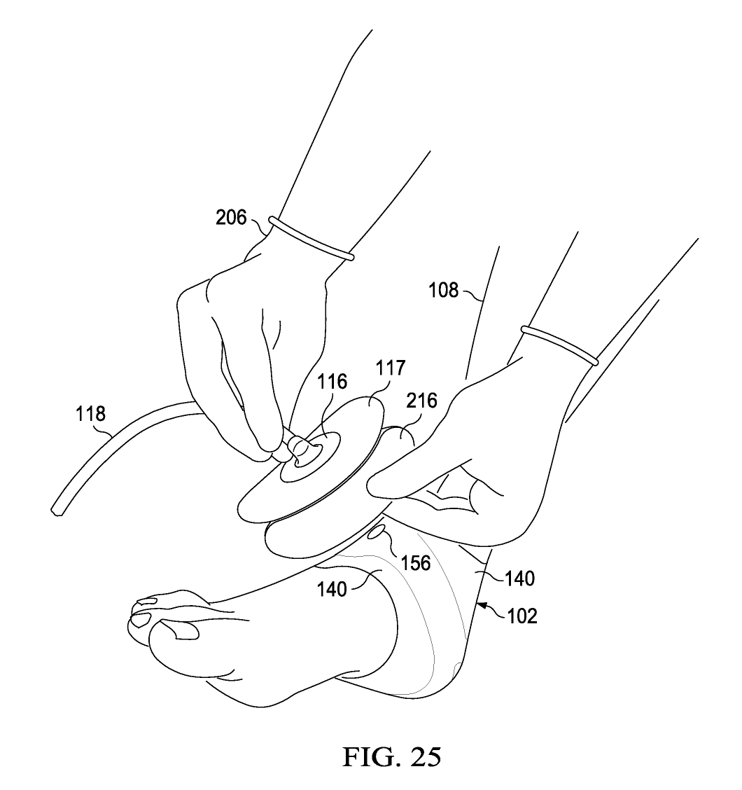

Referring to FIGS. 24 and 25, other components of the reduced-pressure treatment system 100 are shown being installed. The healthcare provider 206 may align the reduced pressure interface 116 and then remove a reduced pressure interface liner 216 to expose an adhesive (not shown) that holds the reduced pressure interface 116 against the outer drape member 140 proximate the fluid aperture 156. The healthcare provider 206 provides pressure and smoothes out around the flange portion 117 of the reduced pressure interface 116. Referring again to FIG. 8, the fully deployed reduced-pressure system 100 is shown. The dressing 102 is shown applied along with other aspects of the reduced pressure treatment system 100 such that treatment may begin on the patient 108.

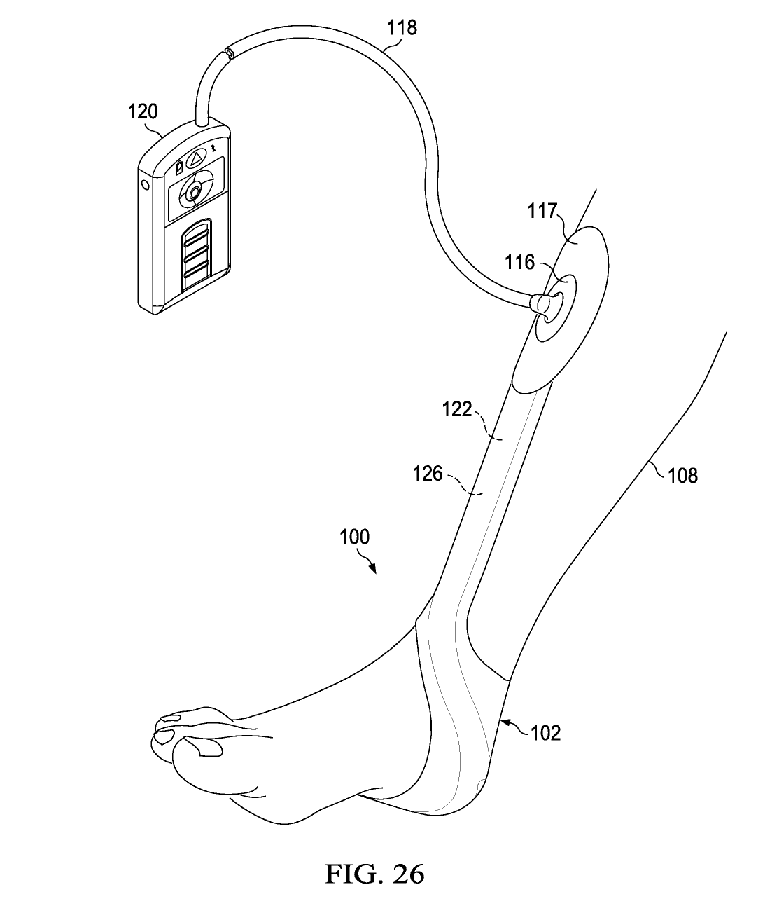

Referring now to FIG. 26, another illustrative embodiment of the reduced pressure treatment system 100 is shown. The system 100 is analogous to that previously presented except for the dressing 102 has a longer extension portion 126 of the transport manifold 122 and the associated parts of the dressing 102. With this embodiment, the reduced-pressure interface 116 may be located further from the wound 110, such as near the patient's knee. This may be advantageous with offloading boots or other treatments. In this embodiment, it may be desirable to form the receiving site release liner 178 in portions. For example, there may be a first receiving site release liner and a second receiving site release liner depending on how much adhesive is desired along the patient's leg. Alternatively, the adhesive 162 may be included just near the pouch 202 and nowhere else on the more proximal portions of the inner drape member 144. In this embodiment, additional adhesive drape strips may be used as desired to secure the dressing 102 to the patient 108.

The embodiment shown in FIG. 26 presents more generally a reduced-pressure bridge that may be used to transport reduced pressure to an extremity such as heel as shown or may be used to transport to reduced pressure along the inside of an off loading boot to a treatment manifold or to a location on a pressure point, e.g., a bed-ridden patient's back. When used merely as a reduced-pressure bridge, the reduced-pressure bridge may be formed without a pouch.

As one illustrative, non-limiting embodiment, the reduced-pressure bridge for delivering reduced pressure to a treatment manifold includes a transport manifold member formed with a receiving site, an extension portion, and application portion. The reduced-pressure bridge further includes an enclosing drape enclosing the transport manifold member. The enclosing drape is formed with an aperture at a proximal end proximate to the receiving site on the transport manifold for the introduction of reduced pressure and at least one aperture at the distal end for egress of reduced pressure. The reduced-pressure bridge has a length (L) and a width (W), and the reduced-pressure bridge has an aspect ratio (L/W) greater than two, three, four, five, six, seven, eight, or more.

The enclosing drape may be formed with an outer drape member and an inner drape member. The outer drape member has a peripheral portion and is sized to have a plan view larger than a plan view of the transport manifold member. The inner drape member has a peripheral portion and is sized to have a plan view larger than the plan view of the manifold member. The manifold member is disposed between the outer drape member and inner drape member. An enclosing attachment may be formed on the peripheral portion of the outer drape member and the peripheral portion of the inner drape member to form a fluid seal between the outer drape member and inner drape member to enclose the manifold member.

Although the present invention and its advantages have been disclosed in the context of certain illustrative, non-limiting embodiments, it should be understood that various changes, substitutions, permutations, and alterations can be made without departing from the scope of the invention as defined by the appended claims. It will be appreciated that any feature that is described in a connection to any one embodiment may also be applicable to any other embodiment.

1. (canceled)

2. (canceled)

3. (canceled)

4. (canceled)

5. A dressing for forming a fluid seal and delivering reduced pressure to a treatment manifold on a wound site of a patient, the dressing comprising:
a transport manifold member formed with a receiving site, an extension portion, and application portion;
an outer drape member having a peripheral portion and sized to have a plan view larger than a plan view of the transport manifold member;
an inner drape member having a peripheral portion and sized to have a plan view larger than the plan view of the manifold member and having at least a portion that is fenestrated;
wherein the manifold member is disposed between the outer drape member and inner drape member;
an enclosing attachment formed on the peripheral portion of the outer drape member and the peripheral portion of the inner drape member to form a fluid seal between the outer drape member and inner drape member to enclose the manifold member;
a pouch-forming attachment formed on a portion of the outer drape member and inner drape member to form a pouch for receiving an extremity of a patient; and
a sealing device coupled to at least a portion of the inner drape member for forming a fluid seal between at least a portion of the inner drape member and a portion of the patient's skin.

6. The dressing of claim 5, wherein the sealing device comprises an adhesive.

7. The dressing of claim 5, wherein the pouch-forming attachment is a weld.

8. (canceled)

9. (canceled)

10. (canceled)

11. (canceled)

12. A dressing for treating an extremity on a patient, the dressing comprising:
a transport manifold having a receiving site, an extension portion, and an application portion;
wherein the application portion of the transport manifold includes a first wing portion and a second wing portion;
an outer drape member having a first wing portion and a second wing portion, and having a proximal end with a fluid aperture formed therein;
an inner drape member having a first wing portion and a second wing portion and having fenestrations proximate the first wing portion and the second wing portion;
an enclosing attachment formed on a peripheral portion of the outer drape member and inner drape member to form an interior portion between the outer drape member and inner drape member;
wherein the transport manifold is disposed within the interior portion; and
a pouch-forming attachment formed at least between the first wing portion and the second wing portion of the inner drape member to form a pouch for receiving the extremity.

13. The dressing of claim 12, further comprising a sealing device disposed on at least a portion of the pouch for forming a fluid seal with the patient's skin.

14. The dressing of claim 12, wherein the first wing portion of the transport manifold includes a first opening and the second wing portion of the transport manifold includes a second opening.

15. The dressing of claim 12, wherein the extension portion of the transport manifold has an aspect ratio greater than four.

16. A system for treating a wound on an extremity of a patient with reduced pressure, the system comprising:
a treatment manifold for disposing proximate the wound;
a dressing comprising:
a transport manifold having a receiving site, an extension portion, and an application portion,
a drape envelope having an outer drape member and a second, patient-facing drape member, further having an interior portion between the outer drape member and the second, patient-facing drape member and having a first wing and a second wing on the drape envelope,
wherein the transport manifold member is disposed within the interior portion, and wherein at least a portion of the second, patient-facing drape member is formed with fenestrations,
a bond formed between the first wing and the second wing on the drape envelope;
a pouch formed by the bond, the pouch for receiving the extremity of the patient, and
a sealing device formed on a portion of the pouch for forming a fluid seal with a portion of the patient's skin; and
a reduced-pressure source fluidly coupled to the transport manifold.

17. The system of claim 16, wherein the sealing device comprises an adhesive.

18. The system of claim 16, wherein the pouch-forming attachment is a weld.

19. A method of treating a wound on an extremity on a patient with reduced pressure, the method comprising:
providing a dressing for treating the extremity, the dressing comprising:
a transport manifold having a receiving site, an extension portion, and an application portion,
a drape envelope having an outer drape member and a second, patient-facing drape member, further having an interior portion between the outer drape member and the second, patient-facing drape member and having a first wing and a second wing on the drape envelope,
wherein the transport manifold member is disposed within the interior portion, and wherein at least a portion of the second, patient-facing drape member is formed with fenestrations,
a bond formed between the first wing and the second wing on the drape envelope;
a pouch formed by the bond, the pouch for receiving the extremity of the patient, and
a sealing device formed on a portion of the pouch for forming a fluid seal with a portion of the patient's skin;
placing the extremity into the pouch;
forming a fluid seal between the pouch and the patient skin around the wound;
attaching a portion of the dressing proximate the receiving site of the transport manifold at a location remote from the wound; and
delivering reduced pressure to the transport manifold.

20. A method of manufacturing a dressing for use on an extremity of a patient, the method comprising:
providing a transport manifold having a receiving site, an extension portion, and an application portion,
forming a drape envelope having an outer drape member and a second, patient-facing drape member, further having an interior portion between the outer drape member and the second, patient-facing drape member and having a first wing and a second wing on the drape envelope,
disposing the transport manifold member within the interior portion;
forming fenestrations on at least a portion of the second, patient-facing drape member;
forming a pouch for receiving the extremity of the patient, wherein forming the pouch includes forming a bond between the first wing and second wing on the drape envelope; and
deposing a sealing device on a portion of the pouch for forming a fluid seal with a portion of the patient's skin.

21. A reduced-pressure bridge for delivering reduced pressure to a treatment manifold, the reduced-pressure bridge comprising:
a transport manifold member formed with a receiving site, an extension portion, and application portion;
an enclosing drape enclosing the transport manifold member, wherein the enclosing drape is formed with an aperture at a proximal end proximate to the receiving site on the transport manifold for the introduction of reduced pressure and an aperture at the distal end for egress of reduced pressure; and
wherein the reduced-pressure bridge has a length (L) and a width (W), and wherein the reduced-pressure bridge has an aspect ratio (L/W) greater than three.

22. The reduced-pressure bridge of claim 21, wherein the enclosing drape comprises:
an outer drape member having a peripheral portion and sized to have a plan view larger than a plan view of the transport manifold member;
an inner drape member having a peripheral portion and sized to have a plan view larger than the plan view of the manifold member and having at least a portion that is fenestrated;
wherein the manifold member is disposed between the outer drape member and inner drape member; and
an enclosing attachment formed on the peripheral portion of the outer drape member and the peripheral portion of the inner drape member to form a fluid seal between the outer drape member and inner drape member to enclose the manifold member.
